(19) United States
(12) Patent Application Publication
(10) Pub. No.: US 2007/0283958 A1
(43) Pub. Date: Dec. 13, 2007

(54) POSITIVE AIRWAY PRESSURE DEVICE

(76) Inventor: Ray Naghavi, Old Westbury, NY (US)

Correspondence Address: DARBY & DARBY P.C., P.O. BOX 770, Church Street Station, New York, NY, 10008-0770, US

(21) Appl. No.: 11/618,641

(22) Filed: Dec. 29, 2006

Related U.S. Application Data
(60) Provisional application No. 60/802,978, filed on May 23, 2006.

(51) Int. Cl. A61M 16/00

(52) U.S. Cl. 128/204.18; 128/204.23

(57) ABSTRACT

A new Continuous/Bi-Level Positive Airway Pressure device (C/BiPAP) is operable to deliver breathing gas such as air, oxygen or a mixture thereof at relatively higher and lower pressures (i.e., generally equal to or above ambient atmospheric pressure) to a patient either as preset or in proportion to the patient's respiratory flow for treatment of Obstructive Sleep Apnea Syndrome (OSAS). The device can be for a single patient or a Dual C/BiPAP having similar elements to the C/BiPAP. A control system can receive inputs for a delta coefficient. The delta coefficient allows a medical professional or the user to designate a stepwise or segmented increase or decrease in pressure from the start of the treatment and/or during the treatment. The C/BiPAP can also include a humidifier, a heater/cooler for the gas, a dehumidifier, a leak detector, a filter to filter allergens and/or dust, an emergency condition detector and a medication chamber.

### RELATED APPLICATIONS

This application claims priority to U.S. Provisional Application Ser. No. 60/802,978 filed May 23, 2006, entitled “NASAL AND ORAL APPLIANCES AND METHOD FOR TREATING SLEEP APNEA.” The contents of which are incorporated herein by reference in its entirety.

### BACKGROUND OF THE INVENTION

1. Field of the Invention

The present invention generally relates to apparatus and methods for treating sleep apnea and/or related breathing disorders. More specifically, the application relates to a positive airway pressure device that has application in the treatment of snoring and obstructive sleep apnea (OSA).

2. Background of the Invention

Sleep apnea is a sleep-related breathing disorder that is thought to affect between 1-10% of the adult population. Recent epidemiologic data indicate that 2% of women and 4% of men between the ages of 30 and 60 years meet the minimum diagnostic criteria for sleep apnea syndrome, representing more than 10 million individuals in the United States. It is a disorder with significant morbidity and mortality, contributing to increased risk of hypertension, cardiac arrhythmias, stroke, and cardiovascular death. Another common sleep-related breathing disorder is snoring, which may be associated with or independent of sleep apnea.

The present invention has been developed to aid in the treatment of snoring and/or the various degrees of hypopnea and apnea that occur due to pathological disturbances in the sleep process. One of the main reasons of the sleep disturbance is the relaxation of the tongue and pharyngeal walls to varying degrees during the several stages of sleep. When fully awake, these tissues have normal tone as air passes in and out of the lungs during respiration. However, during sleep, the musculature supporting these tissues relaxes. As air is inspired, the tongue and posterior walls of the pharynx collapse, causing snoring or, more seriously, causing partial or complete obstruction of the airway.

Obstructive sleep apnea occurs due to a collapse of soft tissue within the upper airway during sleep. The ongoing force of inspiration serves to generate increasingly negative pressure within the pharynx, causing further collapse. The lack of respiration results in inadequate blood oxygenation, and rising carbon dioxide levels. The cardiovascular response produces an increase in the blood pressure and pulse. Cardiac arrhythmias often occur. The carbon dioxide increase and oxygen desaturation triggers a transition to a lighter sleep stage, usually without wakefulness. This transition brings a return to tonicity of the muscles of the upper airway, allowing normal breathing to resume. The person then returns to deeper stages of sleep and the process is repeated. The disease is quantified in terms of respiratory disturbances per hour. Mild disease begins at ten per hour, and it is not uncommon to find patients with indices of about one hundred or more.

Not surprisingly, sleep is extremely fragmented and of poor quality in persons suffering from sleep apnea. As a result, such persons typically feel tired upon wakening and may fall asleep at inappropriate times during the day. All aspects of quality of life, from physical and emotional health, to social functioning are impaired by obstructive sleep apnea.

Continuous Positive Airway Pressure (“CPAP”), disclosed for example in U.S. Pat. No. 5,065,756, is a popular non-surgical treatment for patients suffering from sleep apnea. The disclosure of this patent is incorporated in its entirety herein by reference. CPAP is administered by means of a mechanical unit that delivers pressurized room air to the nasal passage, or airway, through a nose mask that is worn by the patient during sleep. Pressurized air enters from the CPAP unit through the nose when a person is sleeping, and opens the airway from the inside almost as if the air were an internal splint. The correct pressure for the individual is determined in a sleep laboratory. If the nasal airway will admit the flow of air, CPAP has in many cases offered immediate relief. Unfortunately however, compliance with, and long-term acceptance of this treatment are generally poor. Studies have shown that between 20% and 50% of patients fail to use nasal CPAP as prescribed. Problems associated with CPAP include excessive dryness of the mouth and throat, mucous congestion, sinusitis, and rhinorrhea. Breathing against positive air pressure is also discomforting to many patients.

Thus, there is a need for an improved CPAP for more effective treatments for sleep apnea and/or snoring.

### SUMMARY OF THE INVENTION

A new Continuous/Bi-Level Positive Airway Pressure device (C/BiPAP) is operable to deliver breathing gas such as air, oxygen or a mixture thereof at relatively higher and lower pressures (i.e., generally equal to or above ambient atmospheric pressure) to a patient either as preset or in proportion to the patient's respiratory flow for treatment of Obstructive Sleep Apnea Syndrome (OSAS). The device can be for a single patient or a dual/multiple patient C/BiPAP. The Dual/Multiple C/BiPAP has similar elements to the C/BiPAP as described below but capable of generating gas flow for two or more patients from two or more separate gas flow generators.

The C/BiPAP includes a gas flow generator for producing the positive air pressure, such as a conventional CPAP or BiPAP blower (i.e., a centrifugal blower with a relatively steep pressure-flow relationship at any constant speed, compressor, or pump) which receives breathing gas from any suitable gas source. The gas source can be a pressurized bottle of oxygen or air, the ambient atmosphere, an oxygen concentrator or a combination thereof. The gas flow from the flow generator is passed via a delivery conduit to a breathing appliance or patient interface (e.g. a nasal mask, a full face mask, a mouthpiece, or a nasal pillow).

An embodiment includes an adjustable relief valve connected between the gas flow generator and the patient interface. The valve can be mounted by any convenient conventional means at a location separated from the patient and interface. In one embodiment, the C/BiPAP can also include a humidifier (water or ultrasound), a heater and/or cooler for the gas or for the humidified gas, a dehumidifier, a leak detector, a filter to filter allergens and/or dust from the gas flow, and a medication chamber. The de/humidifier can have a selector to allow the patient or medical professional to set the values.

The medication chamber can introduce medication into the airflow passed in the delivery conduit to medicate the patient as they sleep. The medication chamber can introduce a gaseous medication or nebulize a liquid to pass into the airflow. Further, the medication chamber can be used to pass scents into the airflow to have a calming or soothing effect on the patient.

Further, the conduit can have a heating and/or cooling element, to heat or chill the gas or air prior to delivery to the patent. The heating element can be a low voltage coil built into the conduit. The cooling element can be a chiller or a line carrying a refrigeration liquid or gas. The line can run parallel to the conduit to chill the air as it travels. In this, or any other heated/cooled air embodiment using the heater/cooler, the conduit can be insulated. The insulation can be used to retain the temperature in the air stream. Also, the insulation can protect the patient if the heating/cooling element is installed in the conduit. A thermostat can be used to set the temperature to the comfort level selected by the patient. Further, the patient interface can include a thermometer to verify the temperature of the gas as it delivered to the patient to control the temperature accordingly.

The present invention can also include a control system that receives inputs from a medical care provider for at least the proper positive pressure (titrated pressure) for the patient. Alternately, the control system can also be set to “auto titrate” to allow the device to determine the best pressure for the patient. The control system can also control the elements of the C/BiPAP device, for example, the gas flow generator, the humidifier, the dehumidifier, and the leak detector. The inputs can be stored in a memory as well as any information regarding the patient and his or her condition. The inputs can include a set pressure so the device acts as a standard CPAP device. Further, the C/BiPAP includes an electronic circuit to monitor the patient's breathing, and provides two different pressures, a first, higher pressure during inhalation (IPAP) and a second, lower pressure during exhalation (EPAP). Only the IPAP or both the IAPA and the EPAP can be inputted into the control system.

The minimum pressure will, of course, be at least zero and, preferably, a threshold pressure sufficient to maintain pharyngeal patency during expiration. The maximum pressure, on the other hand, will be a pressure somewhat less than that which would result in over-inflation and perhaps rupture of the patient's lungs. Pressures typically range between 5 to 15 centimeters of water.

The electronic circuit can be connected to a flow/pressure sensor such as a flow transducer or similar flow sensing element situated within or near the breathing circuit, i.e., the patient interface, delivery conduit or gas flow generator. The flow sensor may comprise any suitable gas flow meter such as, for example, a bidirectional dynamic mass flow sensor or a pressure responsive sensor for detecting the magnitude of the pressure gradients between the inlet of the patient's airway and his lungs. The flow sensor generates output signals that are fed to the electronic circuitry.

The control system can also receive inputs for a delta coefficient. The delta coefficient allows a medical professional or the patient to designate a stepwise or segmented increase or decrease in pressure from the start of the treatment and/or during the treatment. For example, if a patient's prescribed pressure is 15 mmH₂O, the device can start at a preset pressure and be increased by the delta coefficient until the prescribed pressure is reached, for example 0.5 mmH₂O/minute. During or after the pressure increase or decrease, the C/BiPAP continues to function as a CPAP or BiPAP by either blowing at the incremental pressure or using the incremental pressures as the IPAP pressure.

Alternately, the pressures can be increased over time. For example, the pressure can be set to start at 5 mmH₂O and increase after the first half hour to 10 mmH₂O and decreasing back down after another increment. This allows a lower pressure while the patient is trying to fall asleep and the airway is still supported by the patient's muscles and then increase as the patient enters deeper stages of sleep to the titration pressure. A lower pressure is easier for the patient to exhale against while breathing.

The control system alone or from receiving information from the electronic circuit, the flow sensor and storing information on memory, the patient's compliance with the treatment can be monitored. Pressures, time between uses, changes in settings, and any other information that can be retrieved or that can be helpful to help review the patient's treatment and condition can be recorded to be reviewed by medical professional. The C/BiPAP can also include a communication interface. The communication interface can transmit stored information over the telephone or any network, including a WAN, LAN, and the Internet. Further, medical professionals can enter commands into the control system remotely, once the communication interface is linked to the network. The commands can be passed over a secure network or using any known encryption system to restrict unauthorized changes or access. Additionally, the information can be stored on a smart card or data card and the cards can be mailed to and from patient and medical professional. Additional information can be transmitted over communication interface and/or cards can be received from the medical professional and used to update the control system or the patient settings or monitor patient compliance.

More information, beyond that required for the operation of the C/BiPAP machine, can be transferred by the communication interface. A monitor and interface can be included in the C/BiPAP machine or a connection to a television, personal computer, cell phone or PDA to allow the patient to access to the information on the C/BiPaP device and/or allow the patient and medical professional to communicate. E-mail, text messages and audio/video conferencing and messaging regarding questions and reminders can be transmitted. The patient can have a “face-to-face” video conference with her medical provider to answer questions and the medical provider can send reminders for the next office visit. The communication interface can also communicate with technical support to help initially set up and maintain the C/BiPAP machine.

Additionally, an oxygen meter can be included. In one embodiment, the meter can use focused light to determine the amount of oxygen in the patient's blood stream (also known as oxygen saturation level). The oxygen meter can be connected to the patient's finger or installed in the patient interface. The oxygen meter in the patient interface can take the readings from the patient's nose or mouth using a separate embodiment for an oxygen sensor. The same or a different meter can also detect the patient's pulse. This information (oxygen level and pulse) can be transmitted to the control system and/or memory. The data can be reported back to the medical care provider and/or used to alter the settings for the C/BiPAP device. This can be used to assure that the patient is receiving enough oxygen. Further, this can be linked directly or through the control system to the oxygen concentrator. If a patient's oxygen saturation is low, the concentrator can provide more oxygen to the patient or a valve can be opened to an oxygen bottle.

Furthermore, a CO₂ meter can be installed in the patient interface to help determine the patient's metabolism based on the expired gas and O₂ monitoring. Using this information, weight loss tips can be provided to the patient based on the metabolic analysis. Since many patients requiring a C/BiPAP device are overweight, the C/BiPAP can also provide some advice to cure the disorder and not just alleviate the symptoms.

In addition, the C/BiPaP device can receive inputs based on the patient's age, height, weight, and sex. This information can be used to calibrate the pressures for the C/BiPaP treatments. Additionally, this information can be combined with the O₂/CO₂ meter readings to determine the patient's resting metabolic rate. This indirect calormetry is provided by calculating oxygen consumption by measuring the oxygen inhaled and comparing it to the amount of oxygen exhaled. This comparison is accurate but accuracy can be improved by also measuring CO₂.

Another feature that can be added to the device is a blood pressure cuff 852. Blood pressure cuff 852 can automatically determine the patient's blood pressure. Further, a heart rate monitor and weight determination device can also be included. The weight determination device can be a scale or a body fat scale that can determine both weight and percent body fat. Any or all of this information can be passed to any other system or the control system for patient monitoring.

An emergency condition system, including the leak detector mentioned above, can also be included in the C/BiPAP device. An electric current detector can be disposed to determine if the C/BiPAP device is receiving enough power to continue to operate, or if there are any power fluctuations in the power service. If the electric current detector detects an unusual power condition (i.e. non-power or unstable current) it can sound an alarm to notify the patient that the C/BiPAP device may fail. Further, the C/BiPAP device may have a battery backup or alternate power supply. The battery backup can be triggered once the electric current detector detects the unusual power condition to allow the patient to remain asleep and undisturbed. Furthermore, once the unusual power condition ceases, the C/BiPAP device can be placed back on the normal power supply. The battery backup can also have a visual meter to allow the user to determine the amount of charge remaining in the battery. The battery can be NiCd, Li-ion, zinc-air or standard alkaline batteries, or a combination thereof.

Another emergency system can detect a lack of air being provided to the patient. Once the lack of air is detected, an emergency valve can be opened to allow the patient to take in air from his surroundings, i.e. ambient air. The lack of air detector can be its own unit or can be determined by the control system by referencing the readings from the flow/pressure sensor, the leak detector, and/or the electric current detector. The emergency valve can be biased opened and kept closed under normal operating conditions. In the event of loss of power, the emergency valve can return to its open state without affirmative action from the control system.

Another important consideration is noise from the C/BiPAP device. The use of low noise/vibration pumps and valves as well as insulation can keep the noise level to a minimum. The reduction in noise is a benefit for both the patient and anyone sleeping in the same room as the patient.

Many of the above embodiments can be add-ons to the basic invention. The humidifier, heater, cooler, dehumidifier, filter, communication interface, leak detector, emergency condition detector, oxygen sensor, carbon dioxide sensor, monitor and interface and medication chamber can be added and subtracted as needed by the patient. The basic C/BiPAP can also be a portable device to allow the patient to travel and spend extended time away from home and still use the device. Further, even though the C/BiPaP device is typically used only while the patient is sleeping, the add-ons can be used at any time, day or night, as a complete sleep and patient diagnostic device, such as O₂, CO₂, blood pressure, pulse and weight, and other factors.

### BRIEF DESCRIPTION OF THE DRAWINGS

The above and still further objects, features and advantages of the present invention will become apparent upon consideration of the following detailed description of a specific embodiment thereof, especially when taken in conjunction with the accompanying drawings wherein like reference numerals in the various figures are utilized to designate like components, and wherein:

FIG. 1 is a block diagram of the C/BiPAP device of the present invention; and

FIG. 2 is a block diagram of the Dual C/BiPAP device of the present invention.

### DETAILED DESCRIPTION OF THE INVENTION

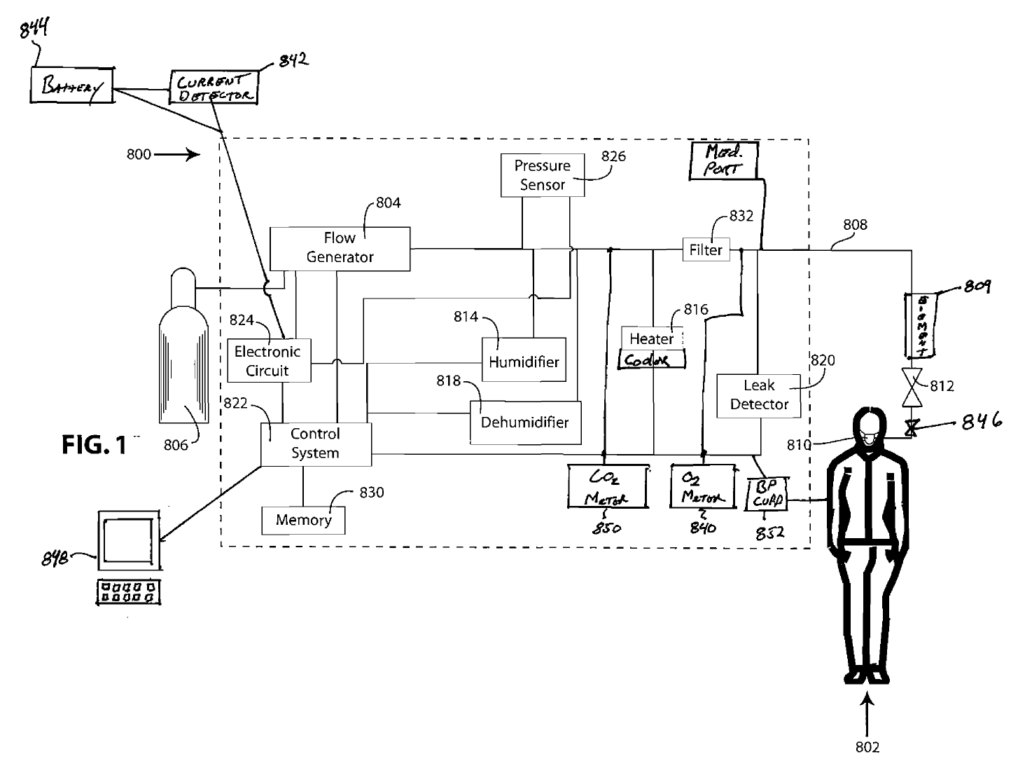

FIG. 1 illustrates a new Continuous/Bi-Level Positive Airway Pressure device (C/BiPAP) 800. C/BiPAP 800 is operable to deliver breathing gas such as air, oxygen or a mixture thereof at relatively higher and lower pressures (i.e., generally equal to or above ambient atmospheric pressure) to a patient 802 either as preset or in proportion to the patient's respiratory flow for treatment of Obstructive Sleep Apnea Syndrome (OSAS).

The C/BiPAP 800 includes a gas flow generator 804 for producing the positive air pressure, such as a conventional CPAP or BiPAP blower (i.e., a centrifugal blower with a relatively steep pressure-flow relationship at any constant speed; compressor or pump) which receives breathing gas from any suitable gas source 806. The gas source can be a pressurized bottle of oxygen or air, the ambient atmosphere, an oxygen concentrator or a combination thereof. The gas flow from flow generator 804 is passed via a delivery conduit 808 to a breathing appliance or patient interface 810 (e.g. a nasal mask, a full face mask, a mouthpiece, or a nasal pillow).

An embodiment includes an adjustable relief valve 812 connected between the gas flow generator 804 and the patient interface 810. The valve 812 can be mounted by any convenient conventional means at a location separated from the patient 802 and interface 810. In one embodiment, the C/BiPAP 800 can also include a humidifier 814 (water or ultrasound), a heater and/or cooler for the gas or for the humidified gas 816, a dehumidifier 818, a leak detector 820, a filter 832 to filter allergens and/or dust from the gas flow, and a medication chamber 834. The de/humidifier can have a selector to allow the patient or medical professional to set the values.

Medication chamber 834 can introduce medication into the airflow passed in the delivery conduit 808 to medicate the patient 802 as they sleep. The medication chamber can introduce a gaseous medication or nebulize a liquid to pass into the airflow. Further, the medication chamber 834 can be used to pass scents into the airflow to have a calming or soothing effect on the patient.

Further, conduit 808 can have heating/cooling element 809, to heat or chill the gas or air prior to delivery to the patent. The heating element can be a low voltage coil built into the conduit 808. The cooling element can be a chiller or a line carrying a refrigeration liquid or gas. The line can run parallel to the conduit to chill the air as it travels. In this, or any other heated/cooled air embodiment using heater/cooler 816, the conduit 808 can be insulated 811. The insulation 811 can be used to retain the temperature in the air stream. Also, the insulation 811 can protect the patient if the heating/cooling element 809 is installed in the conduit 808. A thermostat can be provided to set the temperature to the comfort level selected by the patient. Further, the patient interface 810 can include a thermometer to verify the temperature of the gas as it delivered to the patient 802 to control the temperature accordingly.

The present invention can also include a control system 822 that receives inputs from a medical care provider for at least the proper positive pressure (titrated pressure) for the patient 802. Alternately, the control system can also be set to “auto titrate” to allow the device to determine the best pressure for the patient. The control system 822 can also control the elements of the C/BiPAP device 800, for example, the gas flow generator 804, the humidifier 814, the dehumidifier 818, and the leak detector 820. The inputs can be stored in a memory 830 as well as any information regarding the patient 802 and his or her condition. The inputs can include a set pressure so the device 800 acts as a standard CPAP device. Further, the C/BiPAP includes an electronic circuit 824 to monitor the patient's breathing, and provides two different pressures, a first, higher pressure during inhalation (IPAP) and a second, lower pressure during exhalation (EPAP). Only the IPAP or both the IAPA and the EPAP can be inputted into the control system 822.

The minimum pressure will, of course, be at least zero and, preferably, a threshold pressure sufficient to maintain pharyngeal patency during expiration. The maximum pressure, on the other hand, will be a pressure somewhat less than that which would result in over-inflation and perhaps rupture of the patient's lungs. Pressures typically range between 5 to 15 centimeters of water.

The electronic circuit 824 can be connected to a flow/pressure sensor 826 such as a flow transducer or similar flow sensing element situated within or near the breathing circuit, i.e., the patient interface 810, delivery conduit 808 or gas flow generator 804. The flow sensor 826 may comprise any suitable gas flow meter such as, for example, a bidirectional dynamic mass flow sensor or a pressure responsive sensor for detecting the magnitude of the pressure gradients between the inlet of the patient's airway and his lungs. The flow sensor 826 generates output signals that are fed to the electronic circuitry 824.

The control system 822 can also receive inputs for a delta coefficient 828. The delta coefficient 828 allows a medical professional or the patient to designate a stepwise or segmented increase or decrease in pressure from the start of the treatment and/or during the treatment. For example, if a patient's prescribed pressure is 15 mmH₂O, the device can start at a preset pressure and be increased by the delta coefficient 828 until the prescribed pressure is reached, for example 0.5 mmH₂O/minute. During or after the pressure increase or decrease, the C/BiPAP 800 continues to function as a CPAP or BiPAP by either blowing at the incremental pressure or using the incremental pressures as the IPAP pressure.

Alternately, the pressures can be increased over time. For example, the pressure can be set to start at 5 mmH₂O and increase after the first half hour to 10 mmH₂O and decreasing back down after another increment. This allows a lower pressure while the patient is trying to fall asleep and the airway is still supported by the patient's muscles and then increase as the patient enters deeper stages of sleep to the titration pressure. A lower pressure is easier for the patient 802 to exhale against while breathing.

The control system 822 alone or from receiving information from the electronic circuit 824, the flow sensor 826 and storing information on memory 830, the patient's compliance with the treatment can be monitored. Pressures, time between uses, changes in settings, and any other information that can be retrieved or that can be helpful to help review the patient's treatment and condition can be recorded to be reviewed by medical professional. The C/BiPAP 800 can also include a communication interface 836. The communication interface 836 can transmit stored information over the telephone or any network, including a WAN, LAN, and the Internet. Further, medical professionals can enter commands into the control system 822 remotely, once the communication interface 836 is linked to the network. The commands can be passed over a secure network or using any known encryption system to restrict unauthorized changes. Additionally, the information can be stored on a smart card or data card and the cards can be mailed to and from patient and medical professional. Additional information can be transmitted over the communication interface 836 and/or cards can be received from the medical professional and used to update the control system or the patient settings or monitor patient compliance.

The compliance monitor can be a monitoring system (not illustrated) wherein the data is deposited for automated analysis or future analysis. As part of compliance monitoring, the patient's use of the C/BiPaP device 800 and the specific settings are checked and can be automatically changed or updated by return information to the device.

More information, beyond that required for the operation of the C/BiPAP device 800, can be transferred by the communication interface. A monitor and interface 848 can be included in the C/BiPAP device 800 or a connection to a television, personal computer, cell phone or PDA to allow the patient and medical professional to communicate. E-mail, text messages and audio/video conferencing regarding questions and reminders can be transmitted. The patient can have a “face-to-face” video conference or messaging with her medical provider to answer questions and the medical provider can send reminders for the next office visit. The communication interface can also communicate with technical support, being a human operator or automated system, to help initially set up and maintain the C/BiPAP device 800.

Additionally, an oxygen meter 840 can be included. The meter can use focused light to determine the amount of oxygen in the patient's 810 blood stream (also known as oxygen saturation level). The oxygen meter 840 can be connected to the patient's 802 finger or installed in the patient interface 810. The oxygen meter 840 in the patient interface can take the readings from the patient's 802 nose. Further, the oxygen meter 840 can detect the oxygen from inhaled and exhaled air from the patient 802 and can be in the air stream to do so.

The same or a different meter can also detect the patient's 802 pulse. This information (oxygen level and pulse) can be transmitted to the control system 822 and/or memory 830. The data can be reported back to the medical care provider or used to alter the settings for the C/BiPAP device 800. This can be used to assure that the patient is receiving enough oxygen. Further, this can be linked directly or through the control system to the oxygen concentrator. If a patient's oxygen saturation is low, the concentrator can provide more oxygen to the patient or a valve can be opened to an oxygen bottle.

Furthermore, a CO₂ meter 850 can be installed in the patient interface 810 to determine the patient's metabolism based on the expired gas. Using this information, weight loss tips can be provided to the patient based on the metabolic analysis. Since many patients requiring the C/BiPAP device 800 are overweight, the C/BiPAP device 800 can also provide some advice to cure the disorder and not just alleviate the symptoms.

In addition, the C/BiPaP device 800 can receive inputs based on the patient's age, height, weight, and sex. This information can be used to calibrate the pressures for the C/BiPaP treatments. Additionally, this information can be combined with the O₂/CO₂ meter readings to determine the patient's resting metabolic rate. This indirect calormetry can calculate oxygen consumption by measuring the oxygen inhaled and comparing it to the amount of oxygen exhaled. This comparison is accurate but accuracy can be improved by also measuring CO₂.

Another feature that can be added to the device is a blood pressure cuff 852. Blood pressure cuff 852 can automatically determine the patient's blood pressure and pass that information to any other system or the control system for patient monitoring. Further, a heart rate monitor and weight determination device can also be included. The weight determination device can be a scale or a body fat scale that can determine both weight and percent body fat. Any or all of this information can be passed to any other system or the control system for patient monitoring.

Emergency condition systems, including the leak detector mentioned above, can also be included in the C/BiPaP device 800. An electric current detector 842 can be disposed to determine if the C/BiPaP device 800 is receiving enough power to continue to operate, or if there are any power fluctuations in the power service. If the electric current detector 842 detects an unusual power condition (i.e. non-power or unstable current) it can sound an alarm to notify the patient that the C/BiPaP device 800 may fail. Further, the C/BiPaP device 800 may have a battery backup 844 or alternate power supply. The battery backup 844 can be triggered once the electric current detector 842 detects the unusual power condition to allow the patient to remain asleep and undisturbed. Furthermore, once the unusual power condition ceases, the C/BiPaP device 800 can be placed back on the normal power supply. The battery backup can also have a visual meter to allow the user to determine the amount of charge remaining in the battery. The battery can be NiCd, Li-ion, zinc-air or standard alkaline batteries, or a combination thereof.

Another emergency system can detect a lack of air being provided to the patient. Once the lack of air is detected an emergency valve 846 can be opened to allow the patient to take in air from his surroundings, i.e. ambient air. The lack of air detector can be its own unit or can be determined by the control system 822 by referencing the readings from the flow/pressure sensor 826, the leak detector 820, and/or the electric current detector 842. The emergency valve 846 can be biased opened and kept closed under normal operating conditions. In the event of loss of power, the emergency valve 846 can return to its open state without affirmative action from the control system 822.

Another important consideration is noise from the C/BiPAP device 800. The use of low noise/vibration pumps and valves as well as insulation can keep the noise level to a minimum. The reduction in noise is a benefit for both the patient and anyone sleeping in the same room as the patient.

Many or all of the above embodiments can be add-ons to the basic invention. The humidifier, heater/cooler, dehumidifier, filter, communication interface, leak detector, oxygen sensor, carbon dioxide sensor, blood pressure cuff, heart rate monitor, weight determination device, monitor and interface, medication chamber, etc. can be added and subtracted as needed by the patient. The basic C/BiPAP device 800 can also be a portable device to allow the patient to travel and spend extended time away from home and still use the device. Further, even though the C/BiPaP device 800 is typically used only while the patient is sleeping, the add-ons can be used at any time, day or night, as a complete sleep and/or patient diagnostic device.

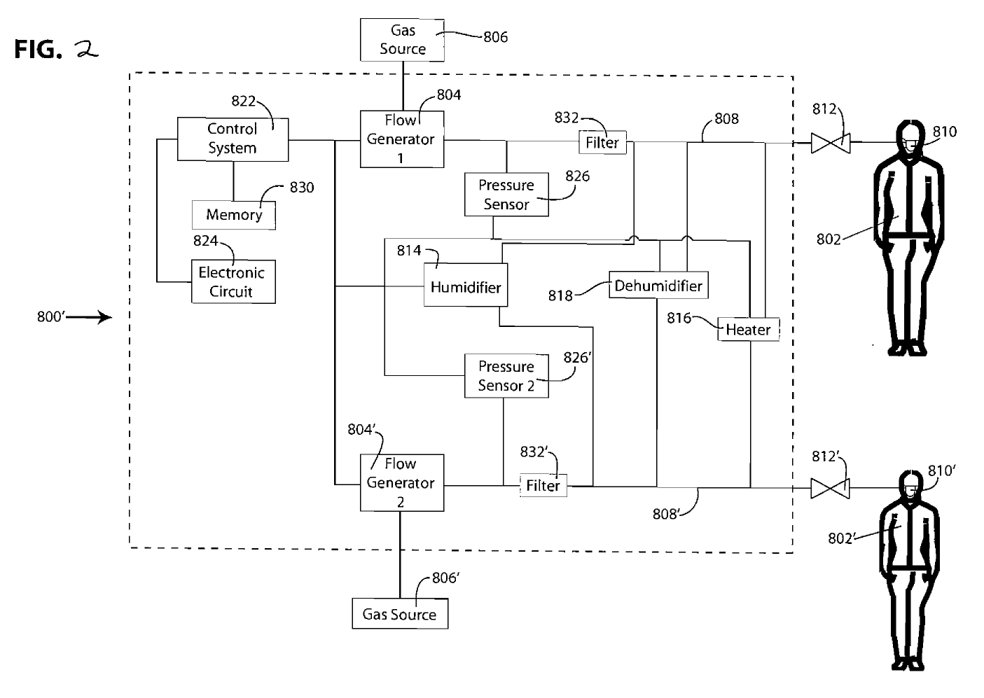

FIG. 2 illustrates a Dual C/BiPAP 800′ having similar elements to the C/BiPAP above but capable of generating gas flow for two patients 810, 810′ from two separate gas flow generators 804, 804′. Each gas flow generator 804, 804′ produces different positive air pressures, each one prescribed for each patient 802, 802′. The gas flow generators 804, 804′ each have their own breathing gas source 806, 806′ (e.g., a pressurized bottle of oxygen or air, the ambient atmosphere, or a combination thereof). Another embodiment utilizes one gas source 806 for both gas flow generators 804, 804′. The gas flow from each flow generator 804, 804′ is passed via its respective delivery conduit 808, 808′ to a breathing appliance or patient interface 810, 810′ (e.g. a nasal mask, a full face mask, a mouthpiece, or a nasal pillow).

An embodiment includes an adjustable relief valves 812, 812′ connected between the gas flow generators 804, 804′ and the patient interfaces 810, 810′. The valves 812, 812′ can be mounted by any convenient conventional means at a location separated from the patients 802, 802′ and interfaces 810, 810′.

In one embodiment, the Dual C/BiPAP 800′ can also include one humidifier 814 (water or ultrasound), heater/cooler 816, dehumidifier 818, and communication interface 836. In an alternate embodiment, each flow conduit 808, 808′ can have its own de/humidifier and heater/cooler. Further, each flow conduit 808, 808′ can have its own a leak detector 820, 820′, filter 832, 832′, and medication chamber 834, 834′.

The control system 822 can control all of the elements of the Dual C/BiPAP device 800′, for example, the gas flow generators 804, 804′ the humidifier 814, the dehumidifier 818, and the leak detectors 820, 820′. All of the inputs listed above can be entered for each patient 802, 802′, and they can be stored in memory 830 as well as any information regarding the patients 802, 802′ and his or her condition.

The electronic circuit 824 can be connected to flow/pressure sensors 826, 826′ such as a flow transducer or similar flow sensing element situated within or near the breathing circuit, i.e., the patient interface 810, 810′, delivery conduit 808, 808′ or gas flow generator 804, 804′. The flow sensors 826, 826′ generate output signals that are fed to the electronic circuitry 824. In another embodiment, the control system 822 can receive inputs for delta coefficients 828, 828′ for each patient 802, 802′.

Thus, while a basic Dual C/BiPAP 800′ device has been described, any of the embodiments described above for the C/BiPAP 800 can be included in one or both breathing circuits. Thus, one circuit can include additional features not found on the other circuit. In this way, each breathing circuit can be tailored to the needs of each specific patient. Another embodiment devises the above embodiments as a modular system. Each “add-on” to the basic C/BiPAP device 800, 800′ is a simple to install add-on so a patient can “upgrade” her device as the need arises or as expenses permits.

Furthermore, additional C/BiPAP devices can be linked to the initial C/BiPAP device to form a dual or even multiple user C/BiPAP device. Multiple C/BiPAP devices can share common additional features if they share the same air source/conduit.

Thus, while there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps which perform substantially the same function, in substantially the same way, to achieve the same results are within the scope of the invention. Substitutions of elements from one described embodiment to another are also fully intended and contemplated. It is also to be understood that the drawings are not necessarily drawn to scale, but that they are merely conceptual in nature.

I claim:

1. A C/BiPAP device for treating a patient, comprising:
a gas flow generator producing a positive air pressure and receiving breathing gas from any suitable gas source;
a patient interface providing the positive air pressure to the patient;
a medication chamber;
a control system controlling the C/BiPAP device; and
a delta coefficient inputted into the control system providing at least one of a stepwise increase or decrease in the positive pressure from the start of the treatment or during the treatment.

2. The C/BiPAP device of claim 1, further comprising an oxygen sensor to determine at least one of a pulse of the patient and the oxygen saturation level of the patient.

3. The C/BiPAP device of claim 2, further comprising an oxygen concentrator communicatively linked to the oxygen sensor.

4. The C/BiPAP device of claim 2, further comprising a carbon dioxide meter to determine at least a metabolic rate of the patient.

5. The C/BiPAP device of claim 1, further comprising an emergency condition system.

6. The C/BiPAP device of claim 5, wherein the emergency condition system comprises a leak detector to determine if air is leaking from the C/BiPAP device.

7. The C/BiPAP device of claim 5, wherein the emergency condition system comprises:
an electric current detector to detect power conditions to the C/BiPAP device; and
a backup power supply to power the C/BiPAP device,
wherein the backup power supply provides power to the C/BiPAP device if the electric current detector detects an unusual power condition.

8. The C/BiPAP device of claim 5, wherein the emergency condition system comprises an emergency valve that opens to provide ambient air to the patient if the emergency condition system detects an emergency condition.

9. The C/BiPAP device of claim 8, wherein the emergency condition system further comprises at least one of a leak detector, a pressure detector, and an electric current detector.

10. The C/BiPAP device of claim 8, wherein the emergency valve is biased opened and kept closed during normal operations of the C/BiPAP device.

11. The C/BiPAP device of claim 1, further comprising at least one of a humidifier, heater/cooler, dehumidifier, filter, communication interface, a monitor and interface and medication chamber.

12. The C/BiPAP device of claim 1, further comprising communication device.

13. The C/BiPaP device of claim 1, wherein the communication device transmits information between the patient and a medical professional or monitoring system.

14. The C/BiPaP device of claim 1 further comprising at least one of a pulse meter, blood pressure device, weight monitor, and heart rate monitor.
